(19) United States
(12) Patent Application Publication
(10) Pub. No.: US 2003/0220544 A1
(43) Pub. Date: Nov. 27, 2003

(54) Method of delivering lubricant for endoscopic procedures

(76) Inventor: Stanley F. Chang, Fresno, CA (US)

Correspondence Address: Richard A. Ryan, RYAN & ENGNATH, 8469 N. Millbrook, Suite 104, Fresno, CA, 93720, US

(21) Appl. No.: 10/153,358

(22) Filed: May 21, 2002

(51) Int. Cl. A61B 1/12

(52) U.S. Cl. 600/156

(57) ABSTRACT

A method for delivering an aqueous lubricating agent into an endoscope during endoscopic procedures, such as colonoscopy. The aqueous lubricant can be prepared by mixing the standard viscous lubricants with an aqueous solution, such as water, or the solution can be supplied in a prepared diluted form. When suctioning debris particles through the channel, the aqueous lubricant can be injected into the channel to break up any clumps of particles and facilitate the suctioning of other coated particles. During an endoscopic procedure when the movement of the endoscope becomes more difficult due to friction, the aqueous lubricant can be injected into the suction/instrument channel of the endoscope to lubricate the area of the body around the distal end of the endoscope. The aqueous lubricant can also be injected into the channel to facilitate passage of medical instruments therethrough.

### BACKGROUND OF THE INVENTION

1. Field of the Invention

The field of the present invention relates generally to procedures for performing endoscopic examinations, such as gastroscopy and colonoscopy. More specifically, the present invention relates to such procedures that include the use of lubricants to facilitate performing a safe and effective endoscopic examination. Even more specifically, the present invention relates to methods of placing lubricants in an endoscope and internal organs to facilitate forward movement of an endoscope through the organs and removal of debris particles from the organs and endoscope.

2. Background

As used herein, the terms “endoscope” or “scope” are used interchangeably to refer to a colonoscope, gastroscope, enteroscope, or other types of medical endoscopes that are used by medical doctors and others to perform medical procedures inside one or more internal organs of a human body. These procedures often involve the passage of other medical instruments through a channel in the scope and removal (i.e., suction) of materials from the internal organ. In the present state of the art, there is one channel for both procedures. The typical endoscope also has a channel for light and a channel for the passage of air or water. For purposes of the present invention, however, the instrument channel is the same as the suction channel and the terms “channel,” “suction channel” and “instrument channel” as used herein all refer to this same suction-instrument channel of the endoscope. In referring to the opposite ends of the scope, the “proximal end” refers to that part of the scope which is closest to the control handle of the endoscope, and the “distal end” refers to that part of the scope farthest from the control handle.

In endoscopic procedures, it is at least highly desirable to advance the endoscope to the furthest point necessary to complete the examination. This is particularly true in colonoscopy, which is the most sensitive and specific means for examining the colon, particularly for the diagnosis of colon cancers and polyps. Because the cecum, the portion of the colon furthest from the anus, is a common location for cancer, it is important that the entire colon be completely examined. However, the technique of total colonoscopy is technically challenging for the practitioner. During a colonoscopy, the scope is inserted in the anus, through the rectum, sigmoid colon, descending colon, transverse colon, ascending colon and then into the cecum. Advancing the scope through the sigmoid can be difficult and painful for the patient due to the turns that result from a loop in the sigmoid colon, stool debris in the colon and friction along the colonic wall. Although colonoscopists have developed techniques, some of which are quite laborious, to work their way through the sigmoid loop, such passage is still difficult primarily due to the generally unaddressed friction between the scope and the colonic wall. Once the sigmoid loop is traversed and then straightened, difficulty arises in trying to keep the sigmoid straight (i.e., keep the sigmoid loop from reforming) to allow reaching the cecum. Devices and procedures, including the use of an overtube or splint, have been developed to address the issue of the reformation of the sigmoid loop (for instance U.S. Pat. Nos. 5,779,624 and 5,941,815 to the present inventor). The present invention provides a method for assisting doctors and technicians with the problems associated with friction and stool debris.

In gastrointestinal endoscopy, the suction channel is utilized for suctioning of undesired fluid and debris material from the lumen of the gastrointestinal tract. In colonoscopy, the debris is most commonly residual stool, which is often present in the colon despite good cleansing preparations. This residual stool obscures the surface of the colon from vision, requiring the debris to be cleaned out, which can be a very laborious, time consuming and frustrating task.

The cleaning process, through the colonoscope, is done by suctioning the stool, with or without injections of water. It would be easy if this debris could be uneventfully and easily suctioned out through the suction channel. However, very often, the solid elements in this debris that are small enough to enter the channel stop moving once inside the suction channel and occlude the suction channel. When this occlusion occurs, the suctioning function is lost, and the endoscopy assistant has to help clear it by injecting air or water through the channel either at the control handle of the endoscope, or, if the occlusion occurs in the long cord connected to the light source, through the end of this long cord. Once the channel is reopened with this maneuver, however, occlusion can reoccur upon resumption of suctioning, such that the process of pushing water through the scope has to be repeated, sometimes many times. The colon is eventually either cleaned out, or this cleaning process is declared a failure. In the latter case, the quality of colonoscopy becomes compromised.

The mechanism of occlusion in the endoscopic channel is caused by friction among the semi-solid debris particles and friction between the stool particles and the inner wall of the suction channel of the endoscope. When suction is applied, particles smaller than the channel size (about 3 mm), enter the channel. Friction resulting from the debris moving against the wall of the channel causes larger particles to slow down during the process of suctioning. The smaller particles, which travel faster due to their size and less friction, being suctioned behind the larger particles catch up with the larger particles. As would be expected based on an understanding of particle adherence, when there is sufficient friction between the two particles, they will adhere to each other to form a clump. As these particles aggregate and become larger and larger particles approximating the size of the channel, they in turn generate more and more friction against the wall of the channel, which causes the clumps to move slow enough that other particles, large and small, will catch up with the clump and form larger clumps. Thus, friction among the debris particles and the friction along the channel wall act in a vicious cycle (both functions of friction).

In addition to stool debris, occluding of the channel can also result from blood clot particles forming larger and larger clumps, whether in conjunction with the stool debris or not. Endoscopists very often have great difficulty with blood clots in cases of acute gastrointestinal bleeding, particularly in the stomach. The blood clots need to be removed from the affected area so the endoscopist can obtain as clear a view as possible of the blood covered areas of the stomach, preferably to the point where the bleeding site can be fully exposed.

With further regard to friction in colonoscopy procedures, as the endoscope moves through the colon it rubs along the colonic wall creating friction that requires the practitioner to apply force to move the endoscope along the colon to perform the desired procedure (the same effect occurs with other types of endoscopic examinations of other internal organs). With the existing endoscopic technique in colonoscopy, the colon is never directly lubricated. Instead, only the endoscope is lubricated while outside the colon, by applying the lubricant (such as K-Y JELLY®), a trademark of Johnson & Johnson Medical Inc. out of Arlington, Tex., and SurgiLube®, a trademark of E. Fougera & Co., a division of Altana, Inc. out of Melville, N.Y.) onto the scope prior to its insertion into the rectum. Some of the lubricant is invariably wiped off upon insertion when the scope slides against the anus, while some remains on the shaft of the scope. As the scope slides against the colonic surface, the lubricant is also gradually and continually rubbed off the scope onto the colon, resulting in a gradient of lubricity starting at the proximal end and declining to the distal end of the scope. The lubricant remaining on the scope and on the colonic surface, in addition to the intrinsic slippery nature of the colonic surface, allows sliding of the scope as it moves against the colonic wall.

As the tip of the scope is advanced further into the colon and away from the rectum, less and less lubricant remains on the distal shaft of the scope, and less and less lubricant is present on the colonic surface that the distal shaft is in contact with. Thus, the closer to the tip of the scope, the less lubricant remaining; the farther away from the rectum, the less the amount of lubricant on the colonic surface. As the scope is advanced toward the cecum, there is more and more length of scope inside the colon, and therefore more and more surface area of the scope in contact with the colonic surface, and thus more and more friction between the scope and the colon. As the lubrication at the distal aspects of the scope becomes more and more depleted, the friction between the endoscope and the colonic wall increases. An improved sliding property of the scope against the colon is obviously desirable. With improved lubrication, the resistance against advancement of the scope is reduced, so that stretching of the colon is also reduced, which in turn reduces pain to the patient.

During medical endoscopy, instruments such as biopsy forceps, polypectomy snares, etc, are frequently utilized. These instruments are passed down the channel of the endoscope, to exit the endoscope at its tip. Sometimes friction is encountered inside the channel, making lubrication of either the inserted instrument or the channel desirable. At present, this is done by applying a drop of silicone lubricant or other lubricant onto the instrument. As with the lubricant applied to the endoscope, the lubricant applied to the instrument tends to be rubbed off as the instrument moves through the channel, particularly at the distal end of the instrument. It would, therefore, be desirable to lubricate the entire channel of the endoscope so as to facilitate the easy insertion and removal of the medical instruments from the endoscope's channel.

What is needed is an improved method of placing lubricants in an endoscope and the internal organs to be inspected or treated so as to facilitate forward movement of an endoscope through the organs. Such an improved method will make it easier to move the endoscope through the body and minimize the amount of labor and patient discomfort associated with an endoscopic procedure, such as a colonoscopy. In addition, such an improved method should be designed to reduce the friction among the debris and blood clot particles and among those particles and the channel wall, thereby reducing the likelihood of clumping inside the channel.

### SUMMARY OF THE INVENTION

The method of delivering lubricant for endoscopic procedures of the present invention provides the benefits and solves the problems identified above. That is to say, the present invention discloses a method of placing lubricants in an endoscope and internal organs to facilitate removal of debris particles from the organs and endoscope channel and forward movement of the endoscope through the organs. The method of the present invention reduces the friction between stool, blood clot and other particles to reduce the formation of larger clumps that occlude the channel. The method of the present invention also reduces the friction of the endoscope and along the channel to facilitate movement of the endoscope through the organs and movement of instruments through the channel. The method of the present invention simplifies endoscopic procedures by reducing the amount of labor required for such procedures and reduces the discomfort of the patient.

To the best of the inventor's knowledge, after a search through available medical resources, the method of lubricating endoscopes and facilitating endoscopic procedures of the present invention has not been previously described. Despite its apparent simplicity, it has never been described before. Generally, if any such improvement did exist it would rapidly spread by word of mouth and in medical publications. To wit, despite the likely demand if this method were previously known to endoscopists, based on its benefits, to date no manufacturer has made commercially available a diluted solution of the aqueous based lubricants, preferably without any potentially undesirable preservatives, suitable for the through-the-scope injection method described herein. The reason this method has not been previously considered is because it is almost counter-intuitive. To most endoscopists, it is almost self-evident that if the fluid in the colon is thickened by lubricants, then suctioning becomes more difficult, hence it is undesirable to inject lubricants through the channel for whatever purpose. However, based on simple principles of physics and from the inventor's own observations, this method is highly effective (i.e., it makes suctioning easier, not harder).

In one embodiment of the present invention, the method of delivering lubricant during a medical procedure using an endoscope includes the steps of inserting the endoscope into a part of the body, such as the gastrointestinal tract, in which the medical procedure is to be performed, advancing the endoscope in the part of the body, placing a quantity of lubricating agent (such as a diluted form of a standard lubricant) into the endoscope under pressure and then advancing the endoscope in the part of the body to perform the desired medical procedure. The method can include the step of providing the lubricating agent prior to inserting the endoscope in the body. The lubricating agent can be provided in a pre-mixed form or by mixing a quantity of the standard, relatively viscous lubricant with an aqueous fluid, such as water, to form a diluted lubricant. The step of providing the lubricating agent, particularly if it is not pre-mixed, can be performed after the endoscope is inserted and then advanced to the point where it becomes known that the lubricating agent is needed. A preferred mechanism for placing the lubricating agent into the endoscope is to inject it into an endoscope channel by using a syringe or squeeze bottle.

As an example, for colonoscopy procedures, the method of the present invention can include the steps of inserting the endoscope into the anus of the colon, advancing the endoscope in the colon, placing a quantity of lubricating agent (such as the diluted lubricant) into a channel of the endoscope under pressure and advancing the endoscope in the colon to perform the desired medical procedure. One preferred embodiment is to inject the lubricant when the tip of the scope is at the upper part of the rectum. Per usual colonoscopic practice, the patient is lying on his or her left side. This will cause the lubricant to flow by gravity through the sigmoid colon to reach the descending colon. The sigmoid and descending colon are thus well lubricated, such that the insertion of the scope through the sigmoid becomes much easier for the practitioner and less painful for the patient.

As with the more general procedure, the lubricating agent can be provided before the procedure is begun by mixing a standard lubricant with an aqueous fluid, such as water, or during the procedure when the need becomes known. Also as with the above, the preferred mechanism for injecting the diluted lubricant into the endoscope channel is a syringe or squeeze bottle.

Accordingly, the primary objective of the present invention is to provide a method of delivering lubricants to the distal end of an endoscope and along the endoscope channel having the features generally described above and more specifically described below in the detailed description.

It is also an important objective of the present invention to provide a method of delivering lubricants to the inside of an internal organ subject to an endoscopic procedure to facilitate removal of debris particles through the endoscope.

It is also an important objective of the present invention to provide a method of delivering lubricants to the inside of an internal organ subject to an endoscopic procedure to facilitate moving the endoscope along the inside of the internal organ.

It is also an important objective of the present invention to provide a method of delivering lubricants to the inside of an internal organ subject to an endoscopic procedure that includes the step of injecting a diluted lubricant into the endoscope channel.

The above and other objectives of the present invention are explained in greater detail by reference to the attached figures and description of the preferred embodiment which follows. As set forth herein, the present invention resides in the novel features of form, construction, mode of operation and combination of parts presently described and understood by the claims.

### BRIEF DESCRIPTION OF THE DRAWINGS

In the drawings which illustrate the best modes presently contemplated for carrying out the present invention:

FIG. 1 is a schematic of the steps of a preferred embodiment of the method of the present invention;

FIG. 2 is a cross-section frontal view of a colon during an endoscopic procedure; and

FIG. 3 illustrates a preferred mechanism for injecting diluted lubricant into the endoscope during a preferred embodiment of the method of the present invention.

### DETAILED DESCRIPTION OF THE PREFERRED EMBODIMENTS

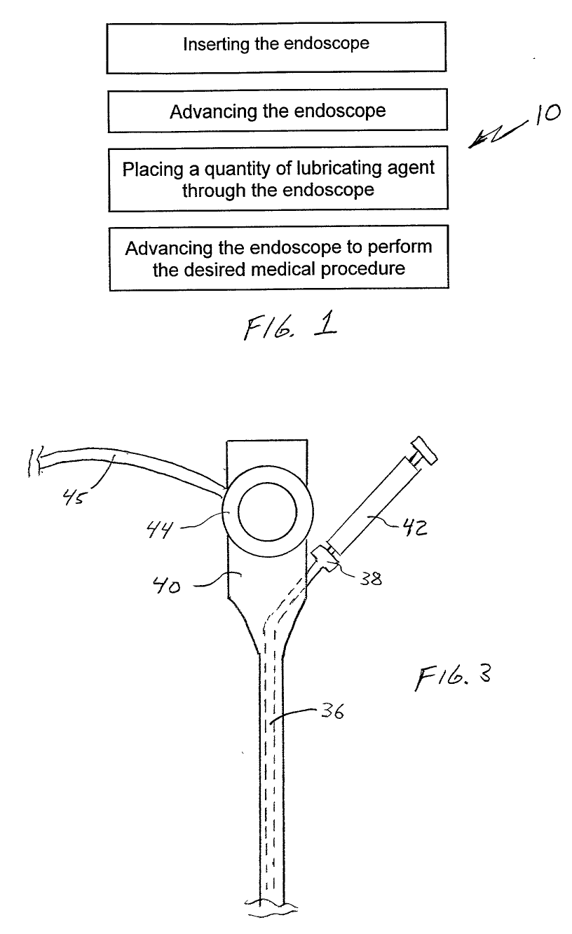

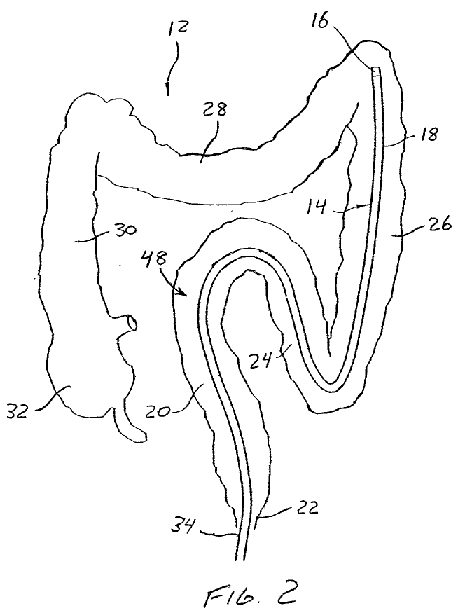

With reference to the figures where like elements have been given like numerical designations to facilitate understanding the present invention, and particularly with reference to the embodiments of the present invention illustrated in FIGS. 1 through 3, the method of delivering lubricant for endoscopic procedure of the present invention is designated generally as 10 in the schematic designated as FIG. 1. During a typical colonoscopy to investigate colon 12, as shown in FIG. 2, the outside of the endoscope (colonoscope) 14 is initially lubricated with standard suitable lubricants, as performed in the prior art. After lubricating endoscope 14 with a first lubricant, the distal end 16 and body 18 of endoscope 14 is introduced into the rectum 20 through the anus 22 and then into the sigmoid colon 24. After negotiating the loop, if any, in the sigmoid colon 24, the endoscope 14 enters the descending colon 26. Once the sigmoid colon 24 is straightened, if necessary, advancement of distal end 16 occurs by further insertion of the endoscope 14 through the transverse colon 28 and ascending colon 30 and then into the cecum 32. Proximal end 34 of endoscope 14 remains outside the anus 22. After completing the desired medical procedure, endoscope 14 is withdrawn from the patient.

The typical endoscope 14 has a channel 36 that is dedicated to the passage of other medical equipment and the suction of debris from inside the organ. As shown in FIG. 3, channel 36 has an injection port 38 on the control handle 40 near the proximal end 34 of scope 14 which is suitable for connecting a syringe 42 or other devices, such as a squeeze bottle (not shown). Also at the proximal end of the scope 14 is a control dial 44 and a connecting cord 45 for connection to a light source device, a machine for suction, and another machine for air and water sprays.

Improved lubrication of the distal part of the endoscope 14 while inside the colon 12 is easily accomplished by injecting a lubricating agent 46 into channel 36 of endoscope 14 to exit at distal end 16 of scope 14 into the colon 12, as best shown in FIG. 3. For purposes of the present invention, lubricating agent is a low viscosity lubricant, such as diluted K-Y JELLY® or SurgiLube® lubricant, that has enough fluidity to flow through channel 36 of scope 14 with relative ease and which contains sufficient amount of the lubricant component to provide the desired lubricating effect described herein. With this method, lubricating agent 46 is delivered to and lubricates the part of the colon 12 that is normally the least lubricated. As known in the art, however, at the present, lubricants used for endoscopy are all sold in their thick and viscous form, most often dispensed through a squeeze tube similar to those used for tooth-pastes and other lotions. This dispenses a thick, paste-like lubricant which is too viscous to allow easy injection through the channel 36 of the endoscope 14.

A lubricating agent 46 suitable for injection through the channel 36 can be prepared by mixing an aliquot of the lubricant (e.g. K-Y JELLY® or SurgiLube®) with an aliquot of water to form an aqueous lubricant 46. This can be done in a clean bowl or other container where thorough mixing can be accomplished by stirring. Another method of formulating the aqueous lubricant is by squeezing some K-Y JELLY® or SurgiLube® into a syringe 42 (shown in use in FIG. 3), which can be done by removing its plunger. After replacing the plunger, water is suctioned into syringe 42. Mixing of the K-Y JELLY®, SurgiLube® or other lubricant is then accomplished by shaking syringe 42. When a pre-mixed aqueous lubricating agent 46, such as a diluted lubricant, becomes commercially available, this step is obviated. The amount of liquidity or dilution for use to clear the channel of debris particles may not be the same amount of liquidity or dilution preferred for lubricating colon 12 for passage of endoscope 14 or for lubricating channel 36 for passage of other medical instruments (not shown). In general, the amount the lubricating agent injected into channel 36 will usually be greater and the lubricant usually more liquid or diluted for debris particle breakup than for lubricating the inside of colon 12 or channel 36.

In use during colonoscopy, when stool debris is encountered in the colon 12, the lubricating agent 46 (i.e., the lubricant-water mixture) is injected through the channel 36 of the endoscope 14 into the stool debris. As shown in FIG. 3, a syringe 42 can be used for injecting the lubricating agent 46 into channel 36. As is well known in the field of medicine, other injection mechanisms that provide a controlled amount of injection under pressures suitable for colon 12 can also be utilized, such as a squeeze bottle that can connect to injection port 38. The stool or blood clot debris in channel 36 will be broken up by the jet of the injected lubricating agent. At the same time, the stool and other debris in channel 36 and colon 12 will be coated by the lubricating agent 46. After injecting an adequate amount of lubricating agent 46, suction is applied to aspirate the stool-lubricant-water mixture into channel 36.

Even when debris in the channel is not a problem, the aqueous lubricating agent 46 can be injected, in a similar manner as above, whenever needed to enhance the movement of the scope against the colon or for improving the lubricious movement of instruments inside the scope channel 36. As stated above, generally the amount of fluid and the degree of dilution may not have to be as great when the objective is to merely lubricate the colon 12 or channel 36. Experience with colonoscopies has indicated that advancement of the scope 14 through the colon 12 is much less painful when lubricating agent 46 is utilized, particularly in the area of the sigmoid colon 24. This is believed to be due to the fact that the pathway of the colonoscope 14 is more friction-free allowing the scope 14 to slide through the tortuosities of the sigmoid loop, resulting in an easier and quicker procedure and less pain for the patient due to reduced stretching of the colon 12 (such as that which results from “forcing” the scope 14 through colon 12 because of friction against the colonic wall).

In one preferred embodiment, the lubricating agent 46 is injected into the sigmoid colon 24 at a location (identified as 48 on FIG. 2) when the tip of the distal end 16 of scope 14 is at the upper part of the rectum. Because, as per usual colonoscopic practice, the patient is lying on his or her left side, the lubricating agent 46 will flow by gravity through the sigmoid colon 24 to reach the descending colon 26. The sigmoid 24 and descending 26 colon are thus well lubricated, such that the insertion of scope 14 through the sigmoid colon 24, where the loop tends to form (as shown in FIG. 2), becomes much easier for the practitioner and less painful for the patient.

While the above description contains many specifics, these should not be construed as limitations on the scope of the invention, but rather as an exemplification of one preferred embodiment thereof. For instance, while this method is particularly pertinent to colonoscopy, it can also be applied to other forms of medical endoscopy. Accordingly, the scope of the invention should be determined not by the embodiments illustrated, but by the appended claims and their legal equivalents.

What is claimed is:

1. A method of delivering lubricant during a medical procedure on a body using an endoscope, comprising the steps of:
a) inserting said endoscope into a part of the body in which said medical procedure is to be performed;
b) advancing said endoscope in said part of said body;
c) placing a quantity of lubricating agent through said endoscope and into said part of said body under pressure; and
d) advancing said endoscope in said part of said body to perform said medical procedure.

2. The method of claim 1 further comprising the step of providing said quantity of lubricating agent prior to said inserting step.

3. The method of claim 2, wherein said providing step includes mixing a quantity of lubricant with an aqueous fluid to prepare said quantity of lubricating agent.

4. The method of claim 3, wherein said aqueous fluid is water.

5. The method of claim 1, wherein said lubricating agent is a lubricant diluted with an aqueous solution.

6. The method of claim 1, wherein said placing step comprises injecting said quantity of lubricating agent into said endoscope.

7. The method of claim 6, wherein a syringe is utilized for injecting said quantity of lubricating agent during said placing step.

8. The method of claim 1, wherein said placing step comprises injecting said quantity of lubricating agent into a channel in said endoscope.

9. A method of delivering lubricant during a medical procedure on a body using an endoscope, comprising the steps of:
a) providing a quantity of lubricating agent;
b) inserting said endoscope into a part of said body in which said medical procedure is to be performed;
c) advancing said endoscope in said part of said body;
d) injecting said quantity of lubricating agent through said endoscope under pressure and into said part of said body; and
e) advancing said endoscope in said part of said body to perform said medical procedure.

10. The method of claim 9, wherein said providing step includes mixing a quantity of lubricant with an aqueous fluid to prepare said quantity of lubricating agent.

11. The method of claim 10, wherein said aqueous fluid is water.

12. The method of claim 9, wherein a syringe is utilized for injecting said quantity of lubricating agent during said injecting step.

13. A method of delivering lubricant during a medical procedure in a colon using an endoscope, comprising the steps of:
a) inserting said endoscope into the anus of said colon;
b) advancing said endoscope in said colon;
c) placing a quantity of lubricating agent into a channel of said endoscope under pressure;
d) forcing said quantity of lubricating agent through said channel and into said colon; and
d) advancing said endoscope in said colon to perform said medical procedure.

14. The method of claim 13 further comprising the step of providing said quantity of lubricating agent prior to said inserting step.

15. The method of claim 14, wherein said providing step includes mixing a quantity of lubricant with an aqueous fluid to prepare said quantity of lubricating agent.

16. The method of claim 15, wherein said aqueous fluid is water.

17. The method of claim 13, wherein a syringe is utilized for injecting said quantity of lubricating agent during said injecting step.
